SEQUENCE LISTING (37 sequences)

SEQ ID NO: 1 - DNA, 405 bp, Aspergillus nidulans
  CDS (1)..(405): basf-dewA hydrophobin
atgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgag

SEQ ID NO: 2 - protein, 135 aa, Aspergillus nidulans
  basf-dewA hydrophobin
MRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAE

SEQ ID NO: 3 - DNA, 471 bp, Aspergillus nidulans
  CDS (1)..(471): basf-rodA hydrophobin
atgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctc

SEQ ID NO: 4 - protein, 157 aa, Aspergillus nidulans
  basf-rodA hydrophobin
MKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSIL

SEQ ID NO: 5 - DNA, 336 bp, Artificial Sequence
  CDS (1)..(336): basf-hypA from chemically synthesized polynucleotide
atgatctctcgcgtccttgtcgctgctctcgtcgctctccccgctcttgttactgcaactcctgctcccggaaagcctaaagccagcagtcagtgcgacgtcggtgaaatccattgctgtgacactcagcagactcccgaccacaccagcgccgccgcgtctggtttgcttggtgttcccatcaaccttggtgctttcctcggtttcgactgtacccccatttccgtccttggcgtcggtggcaacaactgtgctgctcagcctgtctgctgcacaggaaatcaattcaccgcattgattaacgctcttgactgctctcctgtcaatgtcaacctc

SEQ ID NO: 6 - protein, 112 aa, Artificial Sequence
  basf-hypA from chemically synthesized polynucleotide
MISRVLVAALVALPALVTATPAPGKPKASSQCDVGEIHCCDTQQTPDHTSAAASGLLGVPINLGAFLGFDCTPISVLGVGGNNCAAQPVCCTGNQFTALINALDCSPVNVNL

SEQ ID NO: 7 - DNA, 357 bp, Artificial Sequence
  CDS (1)..(357): basf-hypB from chemically synthesized polynucleotide
atggtcagcacgttcatcactgtcgcaaagacccttctcgtcgcgctcctcttcgtcaatatcaatatcgtcgttggtactgcaactaccggcaagcattgtagcaccggtcctatcgagtgctgcaagcaggtcatggattctaagagccctcaggctacggagcttcttacgaagaatggccttggcctgggtgtccttgctggcgtgaagggtcttgttggcgcgaattgcagccctatcacggcaattggtattggctccggcagccaatgctctggccagaccgtttgctgccagaataataatttcaacggtgttgtcgctattggttgcactcccattaatgccaatgtg

SEQ ID NO: 8 - protein, 119 aa, Artificial Sequence
  basf-hypB from chemically synthesized polynucleotide
MVSTFITVAKTLLVALLFVNINIVVGTATTGKHCSTGPIECCKQVMDSKSPQATELLTKNGLGLGVLAGVKGLVGANCSPITAIGIGSGSQCSGQTVCCQNNNFNGVVAIGCTPINANV

SEQ ID NO: 9 - DNA, 408 bp, Schyzophyllum commune
  CDS (1)..(408): basf-sc3 hydrophobin, cDNA template
atgttcgcccgtctccccgtcgtgttcctctacgccttcgtcgcgttcggcgccctcgtcgctgccctcccaggtggccacccgggcacgaccacgccgccggttacgacgacggtgacggtgaccacgccgccctcgacgacgaccatcgccgccggtggcacgtgtactacggggtcgctctcttgctgcaaccaggttcaatcggcgagcagcagccctgttaccgccctcctcggcctgctcggcattgtcctcagcgacctcaacgttctcgttggcatcagctgctctcccctcactgtcatcggtgtcggaggcagcggctgttcggcgcagaccgtctgctgcgaaaacacccaattcaacgggctgatcaacatcggttgcacccccatcaacatcctc

SEQ ID NO: 10 - protein, 136 aa, Schyzophyllum commune
  basf-sc3 hydrophobin, cDNA template
MFARLPVVFLYAFVAFGALVAALPGGHPGTTTPPVTTTVTVTTPPSTTTIAAGGTCTTGSLSCCNQVQSASSSPVTALLGLLGIVLSDLNVLVGISCSPLTVIGVGGSGCSAQTVCCENTQFNGLINIGCTPINIL

SEQ ID NO: 11 - DNA, 483 bp, Artificial Sequence
  CDS (1)..(483): basf-BASF1 from chemically synthesized polynucleotide
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatg

SEQ ID NO: 12 - protein, 161 aa, Artificial Sequence
  basf-BASF1 from chemically synthesized polynucleotide
MKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHM

SEQ ID NO: 13 - DNA, 465 bp, Artificial Sequence
  CDS (1)..(465): basf-BASF2 from chemically synthesized polynucleotide
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccagcagtgcaagcagaacatcgcctgctgccagaactccccttccgacgccaccggctccctcgtcaacctcggcaacccttgcatccctgtctccctcctccatatg

SEQ ID NO: 14 - protein, 155 aa, Artificial Sequence
  basf-BASF2 from chemically synthesized polynucleotide
MKFSVSAAVLAFAASVAALPQHDSAAGNGNGVGNKFPVPDDVTVKQATDKCGDQAQLSCCNKATYAGDVTDIDEGILAGLLKNLIGGGSGSEGLGLFDQCVKLDLQISVIGIPIQDLLNQQCKQNIACCQNSPSDATGSLVNLGNPCIPVSLLHM

SEQ ID NO: 15 - DNA, 882 bp, Bacillus subtilis
  CDS (1)..(882): basf-yaad yaaD
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctgg

SEQ ID NO: 16 - protein, 294 aa, Bacillus subtilis
  basf-yaad yaaD
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGW

SEQ ID NO: 17 - DNA, 591 bp, Bacillus subtilis
  CDS (1)..(591): basf-yaae yaaE with Gly insert at position 2
atgggattaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgtatcaattcatggagccgcttcgtgaattcgctgctcagggcaaaccgatgtttggaacatgtgccggattaattatattagcaaaagaaattgccggttcagataatcctcatttaggtcttctgaatgtggttgtagaacgtaattcatttggccggcaggttgacagctttgaagctgatttaacaattaaaggcttggacgagccttttactggggtattcatccgtgctccgcatattttagaagctggtgaaaatgttgaagttctatcggagcataatggtcgtattgtagccgcgaaacaggggcaattccttggctgctcattccatccggagctgacagaagatcaccgagtgacgcagctgtttgttgaaatggttgaggaatataagcaaaaggcacttgta

SEQ ID NO: 18 - protein, 197 aa, Bacillus subtilis
  basf-yaae yaaE with Gly insert at position 2
MGLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 19 - DNA, 1329 bp, Artificial Sequence
  CDS (1)..(1329): basf-yaad-Xa-dewA-his fusion of Bacillus subtilis yaaD and N-terminal factor Xa proteinase cleavage site and Aspergillus nidulans hydrophobin dewA and his6
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatccattgaaggccgcatgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgagggatctcatcaccatcaccatcac

SEQ ID NO: 20 - protein, 443 aa, Artificial Sequence
  basf-yaad-Xa-dewA-his fusion of Bacillus subtilis yaaD and N-terminal factor Xa proteinase cleavage site and Aspergillus nidulans hydrophobin dewA and his6
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH

SEQ ID NO: 21 - DNA, 1395 bp, Artificial Sequence
  CDS (1)..(1395): basf-yaad-Xa-rodA-his fusion of Bacillus subtilis yaaD and N-terminal factor Xa proteinase cleavage site and Aspergillus nidulans hydrophobin rodA and his6
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctcggatctcatcaccatcaccatcac

SEQ ID NO: 22 - protein, 465 aa, Artificial Sequence
  basf-yaad-Xa-rodA-his fusion of Bacillus subtilis yaaD and N-terminal factor Xa proteinase cleavage site and Aspergillus nidulans hydrophobin rodA and his6
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSILGSHHHHHH

SEQ ID NO: 23 - DNA, 1407 bp, Artificial Sequence
  CDS (1)..(1407): basf-yaad-Xa-BASF1-his fusion of Bacillus subtilis yaaD and N-terminal factor Xa proteinase cleavage site and artificial hydrophobin; BASF1 BASF1 from chemically synthesized polynucleotide
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatgggatctcatcaccatcaccatcac

SEQ ID NO: 24 - protein, 469 aa, Artificial Sequence
  basf-yaad-Xa-BASF1-his fusion of Bacillus subtilis yaaD and N-terminal factor Xa proteinase cleavage site and artificial hydrophobin BASF1; BASF1 from chemically synthesized polynucleotide
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHMGSHHHHHH

SEQ ID NO: 25 - DNA, 561 bp, Artificial sequence
  CDS (1)..(561): DNA sequence encoding fusion protein yaad40-Xa-dewA-his
atggctcaaacaggtactgaacgtgtaaaacgcggaatggcagaaatgcaaaaaggcggcgtcatcatggacgtcatcaatgcggaacaagcgaaaatcgctgaagaagctggagctgtcattgaaggccgcatgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgagggatctcatcaccatcaccatcac

SEQ ID NO: 26 - protein, 187 aa, artificial sequence
  fusion protein yaad40-Xa-dewA-his
MAQTGTERVKRGMAEMQKGGVIMDVINAEQAKIAEEAGAVIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH

SEQ ID NO: 27 - DNA, 28 bp, Artificial Sequence
  Chemically synthesized Hal570 primer
gcgcgcccatggctcaaacaggtactga

SEQ ID NO: 28 - DNA, 28 bp, Artificial Sequence
  Chemically synthesized Hal571 primer
gcagatctccagccgcgttcttgcatac

SEQ ID NO: 29 - DNA, 30 bp, Artificial Sequence
  Chemically synthesized Hal572 primer
ggccatgggattaacaataggtgtactagg

SEQ ID NO: 30 - DNA, 33 bp, Artificial Sequence
  Chemically synthesized Hal573 primer
gcagatcttacaagtgccttttgcttatattcc

SEQ ID NO: 31 - DNA, 38 bp, Artificial Sequence
  Chemically synthesized KaM416 primer
gcagcccatcagggatccctcagccttggtaccagcgc

SEQ ID NO: 32 - DNA, 50 bp, Artificial Sequence
  Chemically synthesized KaM417 primer
cccgtagctagtggatccattgaaggccgcatgaagttctccgtctccgc

SEQ ID NO: 33 - DNA, 45 bp, Artificial Sequence
  Chemically synthesized KaM434 primer
gctaagcggatccattgaaggccgcatgaagttctccattgctgc

SEQ ID NO: 34 - DNA, 30 bp, Artificial Sequence
  Chemically synthesized KaM435 primer
ccaatggggatccgaggatggagccaaggg

SEQ ID NO: 35 - DNA, 38 bp, Artificial Sequence
  Chemically synthesized KaM418 primer
ctgccattcaggggatcccatatggaggagggagacag

SEQ ID NO: 36 - DNA, 32 bp, Artificial Sequence
  Chemically synthesized KaM464 primer
cgttaaggatccgaggatgttgatgggggtgc

SEQ ID NO: 37 - DNA, 35 bp, Artificial Sequence
  Chemically synthesized KaM465 primer
gctaacagatctatgttcgcccgtctccccgtcgt

(19) United States
(12) Patent Application Publication
(10) Pub. No.: US 2011/0192416 A1
(43) Pub. Date: Aug. 11, 2011

(54) USE OF HYDROPHOBIN FOR NON-PERMANENT DYEING OF KERATIN

(75) Inventors: Heiko Barg, Weinheim (DE); Thomas Subkowski, Ladenburg (DE); Marvin Karos, Schwetzingen (DE); Claus Bollschweiler, Heidelberg (DE)

(73) Assignee: BASF SE, Ludwigshafen (DE)

(21) Appl. No.: 13/026,323

(22) Filed: Feb. 14, 2011

Related U.S. Application Data
(63) Continuation-in-part of application No. PCT/EP2009/060537, filed on Aug. 14, 2009.

(30) Foreign Application Priority Data
Aug. 18, 2008 (EP) 08162556.8

(51) Int. Cl. A61K 8/64; A61Q 5/06; A61Q 5/10

(52) U.S. Cl. 132/209; 424/70.6; 8/405

(57) ABSTRACT

The invention relates to the use of hydrophobin for non-permanent dyeing of keratin and keratin-containing materials, in particular of hair, and a corresponding method for non-permanent dyeing of keratin and keratin-containing materials, in particular of hair. The invention further relates to non-permanent dyeing of keratin and keratin-containing materials, in particular, hair, which contain hydrophobin.

### RELATED APPLICATIONS

This application is a continuation-in-part of PCT/EP2009/060537, filed Aug. 14, 2009, which claims benefit of European application 08162556.8, filed Aug. 18, 2008, both of which are herein incorporated by reference in their entirety.

### SUBMISSION OF SEQUENCE LISTING

The sequence listing associated with this application is filed in electronic format via EFS-Web and hereby incorporated by reference into the specification in its entirety. The name of the text file containing the Sequence Listing is Sequence_Listing_{—}13477_{—}00021_US.txt. The size of the text file is 75 KB, and the text file was created on Feb. 11, 2011.

### BACKGROUND OF THE INVENTION

The present invention relates to the use of hydrophobin for non-permanent dyeing of keratin and keratin-containing materials, in particular of hair, and a corresponding method for non-permanent dyeing of keratin and keratin-containing materials, in particular of hair. The invention further relates to means of non-permanent dyeing of keratin and keratin-containing materials, in particular, hair, which contain hydrophobin.

Hair dyeing compositions or hair coloring compositions are classified according to the fastness. Various classifications are known. In most cases a sub-classification in three classes is made: temporary hair dyeing compositions (colorations in a narrow sense), which last for only 1-2 hair washes (Class 1), semi-permanent hair dyeing compositions, that have to be renewed after about 6-10 hair washes (Class 2) and permanent hair dyeing compositions (hair dyeings in a narrow sense), which may not be washed out (Class 3). In another, equally common sub-classification, every dyeing composition that may be washed out and lasts for up to 10 hair washes are subsummed as colorations (Class 1), whereas Class 2 is attributed to demi-permanent hair dyeings, which lasts for up to 24 hair washes, while Class 3 also designates permanent hair dyeing compositions. Furthermore, a sub-division into four classes is also found in the literature: colorations (1-2 hair washes), semi-permanent dyeing (6-10 hair washes), demi-permanent dyeings (up to 24 hair washes) and permanent dyeing (not washed out).

The compositions in Classes 1 and 2 according to all classifications have in common that they may be washed out, accordingly they are herein below subsummed as referring to as dyeings that may be washed out or that are non-permanent. Semi- and demi-permanent dyeings are subsummed herein below as dyeings of Class 2, whereas dyes that may be washed out after only 1-2 hair washes refer to colorations of Class 1.

All commercial products of Class 3 for the permanent dyeing of hair on the one hand contain a “developer”, generally an oxidizing chemical, and on the other hand an alkaline component as part of the dyeing paste, usually ammonia or an ammonia substitute such as, e.g. mono-ethanolamine. These compounds have the function of rendering the hair fiber cuticula permeable for the colorless dye precursors, facilitating the development of the final dyes inside the hair and at the same time bring out a lightening through the influence of the peroxide. As soon as the dyeing paste containing the alkaline ingredient is brought into contact with the developer, hydrogen peroxide is deprotonated, diffuses through the cuticula and reaches the interior of the hair, where the melanin is found. The alkaline peroxide destroys the melanin and oxidizes the initially colorless precursors of the dye yielding the final dye molecules, which are too large to exit the hair. Due to this chemical process, permanent hair dyeing compositions are also referred to as oxidizing hair dying compositions. In principle, so called “self-oxidizing dyes” which are oxidized by atmospheric oxygen belong to the permanent hair dyeings.

Temporary and semi-permanent hair dyeing compositions (hereinafter summarized as non-permanent dyeing compositions) generally do not function non-oxidatively. The molecules of the dye arrange themselves on the keratin surface or they penetrate the hair only for a marginal distance. They are too large to completely permeate the hair. The so-formed dye layer can be removed by washing with hair shampoo.

In general, acidic dyes with only low affinity for hair and which rather deposit on the hair surface are used in temporary hair dyeing compositions (synonym: colorations, direct dyeing compositions) of Class 1.

Dyes in colorations are generally either positively charged and therefore bind to negatively charged surface groups of the hair, or they are small molecules that can penetrate the cuticula.

Temporary hair dyes do not penetrate the hair shaft itself. Instead the dyes remain bound to the cuticula and can often be removed in a single hair wash.

By contrast, semi-permanent hair dyes (which are often commonly referred to as colorations, intensive colorations or direct dying agent) can penetrate into the hair, because they contain smaller dye molecules than temporary hair colorations. Accordingly, semi-permanent hair dyeings sustain longer than temporary colorations and generally have to be renewed after only 8 to 10 hair washes. Demi-permanent dyeings, the dyes of which penetrate even better into the hair, last considerably longer, in most cases for up to 24 hair washes.

Based on their rinsability, both semi- as well as demi-permanent dyeings are referred to by the manufacturers as colorations, often also as colorations of Class 1 although it would be more appropriate to classify them into Class 2.

Hair dyeing compositions are usually sold in form of aqueous solutions or emulsions, which are as concentrated as possible and contain, in addition to the actual dyes, for example fatty acid alcohols and/or other oily components, emulsifiers as well as surface-active agents and optionally alcohols. Colorations and semi-permanent hair dyeing compositions are available in form of various products, inter alia, as pastes, conditioners, shampoos, gels and sprays.

Non-permanent hair dyeing compositions have a smaller share to the market than permanent hair dyeings. Nonetheless, they are of important economical interest, because they affect the hair less than permanent hair dyeings and contain neither bleaching agents nor ammonia. In addition, due to some of their ingredients, in particular hydrogen peroxide and ammonia, permanent hair dyes have been criticized for some time. In the EU, permanent dyes which have not been examined with respect to their compatibility will be prohibited in the future.

Furthermore, permanent hair dyeing compositions affect the structure of the hair, since they have to permeate the protective layer of the hair. In addition, permanent hair dyes are suspected to cause cancer of the bladder (A. Andres: Int J Cancer 2004; 109: 581-586).

This has caused an increasing interest in non-permanent alternatives to permanent hair dyeing compositions. However, the consumer often considers it as a disadvantage that colorations last only for a single hair wash and that dyeings of Class 2 fade out after a few washes.

Accordingly, there is a need for non-permanent hair dyeing compositions and corresponding methods, which are alternatives or improvements of already existing compositions and methods in the non-permanent coloration of hair, and which, in particular, allow for a more stable adherence of the dyes to the hair, if possible without concomitantly damaging the hair.

Hydrophobins are a class of small, cysteine-rich proteins of about 100-150 amino acids in length, which occur in nature only in filamentous fungi. They are amphiphilic and may form a water-insoluble layer on the surface of an object. Their natural functions comprise the coating of fungal spores to prevent them from sticking to each other, the coating of air hyphae to reduce the surface tension of water thus making the uptake of water easier, and possibly transmission of signals between a fungus and its environment (Whiteford, J. F. Spanu, P. D. (2001), Fungal Genet. Biol. 32(3): 159-168; Wösten et al. (1999) Current Biol. 19: 1985-88; Bell et al. (1992), Genes Dev. 6: 2382-2394).

In 1999, the first discovery and purification of hydrophobin was made in Schizophyllum commune. Meanwhile, hydrophobin-genes have been identified in ascomycetes, deuteromycetes and basidiomycetes. Various fungi contain more than one hydrophobin gene, e.g. Schizophyllum commune, Coprinus cinereus and Aspergillus nidulans.

On the basis of differences in hydrophobicity and biophysical properties of the hydrophobins they were classified into two categories: class I and class II. Complementation experiments have shown that, up to a certain extent, hydrophobins of one class may functionally replace hydrophobins of the other class. The various hydrophobins appear to be involved at different developmental stages in fungi and seem to exert different functions therein (van Wetter et al. (2000) Mol. Microbiol. 36:201-210; Kershaw et al. (1998) Fungal Genet. Biol. 23: 18-33).

### DESCRIPTION OF RELATED ART

In general, hydrophobins have eight cysteine-units. They may be isolated from natural sources, but they may also be obtained by genetic engineering processes, as described for example in WO 2006/082251 and WO 2006/131564.

The use of hydrophobins in cosmetic preparations is known per se. US 2003/0217419 A1 describes the use of the hydrophobin SC3 of S. commune for the treatment of keratin-containing materials. Thereby, cosmetic depots are formed that should resist several washings with shampoo. The hydrophobin is applied either concomitantly with or subsequent to the cosmetically active ingredient, but not before applying the cosmetically active ingredient.

WO 2006/136607 A2 describes the use of hydrophobin and of hydrophobin-conjugates in cosmetic preparations for hair care. According to WO 2006/136607 hydrophobins may be coupled to semi-permanent or permanent hair dyes and increase their concentration and effect on skin and hair. In the case of permanent hair dyes one of the two dye-components is bound to hydrophobin, while the other component is added after applying the conjugate to the hair. The oxidative coupling of both components then occurs directly on the hair. The use of hydrophobin together with temporary hair dyeing compositions of Class 1 is not described in WO 2006/136607.

WO 2006/082251 describes hydrophobin-proteins, their production and their use in the coating of surfaces.

WO 96/41882 suggests using hydrophobins, inter alia, as surface active substances in order to render hydrophobic surfaces hydrophilic, for the improvement of water-resistance of hydrophilic substrates, and in the manufacture of hair shampoos and conditioners.

### OBJECT OF THE INVENTION

The object of the present invention is to provide methods and compositions to support non-permanent dyeings, in particular the coloration or the semi-permanent dyeing, of keratin and keratin-containing materials (such as e.g., hair, skin, nails, but also wool, leather and other keratin-containing textiles), particularly of hair.

A further object is the provision of a method for dyeing of keratin, in particular for dyeing hair, wherein the keratin fiber is damaged as little as possible. In accordance with the present invention, the dyeing of keratin should preferably be non-permanent, but it should exceed currently available non-permanent, in particular temporary hair dyeings in view of their fastness and/or color intensity.

It is a further object to improve the dyeing of keratin achieved by conventional non-permanent dyeing compositions, in particular to increase the color intensity and/or the fastness of the coloration or the dyeing against washing out, without making the application of oxidizing dyes necessary. Preferably, this goal is strived after for colorations and semi-permanent dyeings. Thereby an alternative to permanent dyeings, which is not associated with the disadvantages of these dyeing compositions, should be established.

These and other objects, which may be derived from the description of the invention herein below, are solved according to the independent claims. Specific embodiments of the invention can be derived from the dependent claims, the description and the examples. Furthermore, the invention comprises also combinations of these preferred embodiments.

### BRIEF SUMMARY OF THE INVENTION

The present invention relates to the use of hydrophobin and hydrophobin-containing compositions for the non-permanent dyeing of keratin or keratin-containing material, particularly of hair. The invention further relates to a corresponding composition for dyeing of keratin, specifically a coloration or semi-permanent dyeing. A corresponding method for dyeing of keratin using hydrophobin is equally presented.

The use of hydrophobin preferably results in an increased color intensity and longer wash-out period of the dyeing, which suggests an improved uptake and/or deposition of non-permanent dyes in/on the keratin. This concerns particularly hydrophilic dyes. The dyeing is thus rendered more intensive and/or longer lasting.

The use of hydrophobin can further lead to an improved uptake and/or deposition of other cosmetic agents (in addition to the non-permanent dyes) in/on the keratin. This concerns preferably hydrophilic cosmetically active agents, such as panthenol. Thereby, the active agents act more intensively, because a higher local concentration thereof reaches and/or penetrates the keratin and/or the period until these agents are completely washed out is extended. Amongst others, this can have an effect on the hair thickness, the tear strength (tear force improvement), combability, combing force, lissomness and other properties of the treated keratin. The individual cosmetic agent and the field of application determines respective quantitative and qualitative effects.

In particular due to the presence of hydrophobin, additional components of colorations/dyeings, apart from the dyes, e.g. conditioning agents, may act more intensively.

### DETAILED DESCRIPTION OF THE INVENTION

In detail, the present invention relates to the following subject matter and embodiments, respectively:
(1) a method for non-permanent dyeing of keratin or keratin-containing material, comprising the application of at least one non-permanent dye and at least one hydrophobin of structural formula (1) on the keratin or keratin-containing material;
(2) a composition for non-permanent dyeing of keratin or keratin-containing material, containing
   (i) at least one hydrophobin of structural formula (I), and
   (ii) at least one non-permanent dye;
(3) a kit for non-permanent dyeing of keratin or keratin-containing material, comprising two separate cosmetic compositions, i.e.
   (i) a composition containing at least one hydrophobin of formula (I), and
   (ii) a composition containing at least one non-permanent dye; and
(4) the use of a hydrophobin of structural formula (I) for non-permanently dying of keratin or keratin-containing material to increase the intensity of the non-permanent dyeing and/or the fastness of the non-permanent dyeing against washing out.

### DEFINITIONS

The following terms, definitions and abbreviations are used:

Conventional three- or single-letter codes for amino acids and nucleotides.

Within the context of the present invention the singular form “a(n)” encompasses also the corresponding plural, inasmuch as the context does not result in something different. Thus, the term “a hydrophobin” may also comprise more than one hydrophobin-molecule, i.e. two, three, four, five, etc. hydrophobins of a single type.

“At least one” means “one or more”, “at least” followed by a numerical value means “this or a higher numerical value”.

The term “about” or “ca.” in context of a numerical value or within the limits of an area of parameter designates an area of deviation, wherein in accordance with the understanding of the skilled person in the art the technical effect of the feature in question is still assured. Typically, the term means a deviation of +/−10%, preferably +/−5% from the indicated numerical value.

If not indicated to the contrary, acids are present in their free form, either as free acid or as partial or complete salt of said acid or as a mixture of the acid and its salt. Conversely, bases, particularly amines, may be present in form of a free base or as a partial or complete salt of said base or as a mixture of the base and its salt.

“Native” is a synonym for “wildtype” and “naturally (occurring)”. A “naturally” occurring bond between two polypeptides is a bond, which is as it is found in nature, that is, for example, in a wildtype protein. A wildtype or a native protein or polypeptide is, provided there are no indications to the contrary, the usually occurring form of the said protein/polypeptide.

In the context of the present invention “recombinant” means “produced with the aid of or as a result of genetic engineering methods”.

A “fragment” of an amino acid sequence results from the lack of one or more successive amino acids at the N- and/or C-terminus of the respective original sequence.

A “homolog” of an amino acid sequence in accordance with the present invention is a protein or polypeptide, which differs from the original sequence by substitution of one or more amino acids. Preferably the function and/or conformation of the protein is not influenced by said substitution. Particularly preferable, the amino acid substitution is a conservative amino acid exchange, the exchanged amino acids are thus replaced by amino acids having similar chemical properties, e.g. Val by Ala.

Particularly preferable conservative amino acid exchanges occur between members of the following groups:
(1) acidic amino acids (aspartic acid and glutamic acid);
(2) basic amino acids (lysine, arginine, histidine);
(3) hydrophobic amino acids (leucine, isoleucine, methionine, valine, alanine);
(4) hydrophilic amino acids (serine, glycine, alanine, threonine);
(5) amino acids having aliphatic side chains (glycine, alanine, valine, leucine, isoleucine);
(6) amino acids having hydroxyaliphatic side chains (serine, threonine);
(7) amino acids having amide-groups in the side chain (asparagine, glutamine);
(8) amino acids having aromatic side chains (phenylalanine, tyrosine, tryptophane);
(9) amino acids having a sulfur in the side chain (cysteine, methionine).

Especially preferred conservative amino acid exchanges are:

| Original amino acid | substituent |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term “isolated” means “separated or purified from the original organism”. Accordingly, an isolated hydrophobin is no longer part of the fungus wherein it is found in nature. Recombinantly produced hydrophobins are also “isolated” hydrophobins.

“Color intensity” (synonym: “intensity of dyeing”) is the observed colorfulness that may be either perceived by subjective assessment based upon visual inspection (optionally in comparison with an non-dyed standard), or which may be determined in a comparison of optical measurement data.

Such measurement data may for example be determined with a conventional photometer or colorimeter, such as the Konica Minolta CR-300, CR-310, CR-311 (Konica Minolta Sensing, Inc., Osaka, Japan), preferably using the CR-300. The measurement data may then, for example, be evaluated in accordance with the a-b-L-System (synonym: Lab-System, CIELAB-System; L: brightness; a: tonality; b: saturation) or in accordance with the L*a*b*-system (CIE 1976), preferably in accordance with the L*a*b*-System. The evaluation according to the L*a*b*-system is described, e.g. in the instructions manual of the Konica Minolta CR-300, CR-310, CR-311 (Konica Minolta Sensing, Inc., Osaka, Japan), German version, version number 527 349/9.99.

The term “non-permanent dye” designates a dye, whose dyeing effect on the keratin may be reversed by a single or by repeated washings, i.e. a dye for non-permanently dyeing of keratin.

In the context of the present invention, the terms “non-permanent/washable dyeing” and “non-permanent/washable (hair)dyes”, respectively, comprise colorations, semi- and demi-permanent dyeings and dyes for keratin, that is any dyeings and dyeing compositions that may be washed-out from the keratin and that are not permanent hair dyes, respectively.

In the context of the present invention, the term “coloration” is used as a synonym for “temporary coloration”, “temporary dye”, “temporary color”, “temporary dyestuff”. It comprises, due to the inconsistent linguistic usage by the manufacturers (supra, Background), in its broader sense any non-permanent, washable hair dyeing compositions, furthermore temporary dyeing compositions (colorations in the proper meaning of the word) semi- and demi-permanent dyeing compositions.

In the narrower, preferred sense it comprises colorations and semi-permanent dyeing compositions, in the most preferred and the narrowest sense it comprises colorations in the proper meaning of the word. In the proper meaning of the word, a coloration is a dying composition for non-permanently dyeing of hair that may be washed out within 1-2 hair washes. Further definitions and explanations are found in the section “background”.

“ΔE” (synonym: “Delta E”) is a measure for the difference in color and the color distance (DIN 5033-1: 1979-03), respectively, between a sample color and a comparative color as well as between two colors. ΔE may be determined photometrically according to different industry standards, conventionally by measurement in accordance with the standard DIN5033 and the calculation of ΔE pursuant to DIN 6174. The measurement data may be evaluated in accordance with the a-b-L-System (synonym: Lab-System, CIELAB-System; L: brightness; a: tonality; b: saturation) or according to the L*a*b*-system (CIE 1976). A preferred method is based on the L*a*b* color space defined in CIE 1976. For the purpose of the present invention, ΔE is preferably determined according to the standard DIN 5033-Part 1 and DIN 6174, namely on the basis of the L*a*b*-color space, e.g. with the aid of a colorimeter such as the Konica Minolta CR-300, CR-310, CR-311 (Konica Minolta Sensing, Inc., Osaka, Japan), preferably with the CR-300, and in accordance with the method described in the instructions manual of the Konica Minolta CR-300, CR-310, CR-311 (Konica Minolta Sensing, Inc., Osaka, Japan), German version, version number 527 349/9.99 in accordance with the method described in Example 6.

As a rule, the ΔE-values of perceptible color differences are between 2 and 5, in case of a very good result they are above 5, which indicates a conspicuous color difference visible to the naked eye (the presence of a different color):

| ΔE | Rating |
|---|---|
| 0.0 . . . 0.5 | no or almost no difference |
| 0.5 . . . 1.0 | difference may be noticeable to the trained eye |
| 1.0 . . . 2.0 | noticeable color difference |
| 2.0 . . . 4.0 | perceptible color difference |
| 4.0 . . . 5.0 | substantial color difference, which is rarely tolerated |
| Above 5.0 | the difference is rated as a different color. |

The terms “hydrophilic” and “hydrophobic” have the meanings conventionally attributed in chemical terminology. Thus, a “hydrophilic” dye is a dye that is preferably soluble in water or polar solvents. Hydrophilic dyes are typically polar compounds that are either ionic, have a dipole moment and/or contain electronegative groups. On the other hand, a “hydrophobic” dye dissolves preferably in non-polar media and does not have ionic functional groups and only weak electronegative functional groups.

### DETAILED DESCRIPTION OF THE INVENTION

The present invention relates to the use of hydrophobin for non-permanent dyeing of keratin and keratin-containing materials as well as corresponding hydrophobin-containing cosmetic compositions and kits.

It has surprisingly been found that the intensity of non-permanent dyeing and/or the fastness of non-permanent dyeing against washing-out is increased upon use of hydrophobin in accordance with the invention, in particular in the use in a method pursuant to embodiment (1), compared with non-permanent dyeing without use of said hydrophobin. This applies in particular to a pre-treatment of the keratin to be dyed with a composition containing hydrophobin prior to the application of the non-permanent dye. The color difference between hydrophobin-treated and—untreated dyed hair, expressed as ΔE and preferably determined in accordance with DIN 5033 and/or calculated according to DIN 6174, e.g. according to the method described in the instructions manual of the Konica Minolta CR-300, CR-310, CR-311 (Konica Minolta Sensing, Inc., Osaka, Japan), German version, version number 527 349/9.99 (see the section “Definitions” and Ex. 6), may here be more than 2, preferably more than 4, particularly preferred more than 5. The effect of the hydrophobin is possibly based on the fact that hydrophobin can increase the hydrophilicity of a keratin-containing surface, in particular the surface of hair.

The method according to embodiment (1), the compositions according to embodiments (2) and (3) and the use according to embodiment (4) preferably serve only for cosmetically dyeing of keratin and therefore are not therapeutic treatments of the human or animal body.

The subject matter of the present invention serves in the dyeing of keratin. The keratin may either be present as pure keratin or it is part of keratin-containing materials. Preferred keratins and keratin-containing materials are skin, hair, nails, horn, wool, leather, coat, fur and feathers. Particularly preferred are keratinaceous fibers, in particular hair and wool.

Especially preferably, the method according to embodiment (1) is used for the dyeing of hair, in particular of hair of the scalp, and the compositions according to embodiment (2) and (3) as well as the use (4) are suitable for dyeing of hair, in particular hair of the scalp.

The keratin that is dyed according to the invention may be of human or animal origin and is preferably of human origin.

The method according to embodiment (1) and the use according to embodiment (4) of the present invention requires the application of at least one hydrophobin on the keratin to be dyed.

The at least one hydrophobin is preferably a component of a composition, which contains additional cosmetically acceptable ingredients.

In the context of every embodiment of the present invention, the terms “hydrophobin” and “hydrophobins”, respectively, designate preferably polypeptides according to general formula (I)

Xₙ—C¹-x₁₋₅₀-C²—X₀₋₅—C³—X₁₋₁₀₀—C⁴—X₁₋₁₀₀—C⁵—X₁₋₅₀—C⁶—X₀₋₅—C⁷—X₁₋₅₀—C⁸—Xₘ (I),

wherein X may stand for each of the 20 naturally occurring amino acids (Phe, Leu, Ser, Tyr, Cys, Trp, Pro, His, Gln, Arg, Ile Met, Thr, Asn, Lys, Val, Ala, Asp, Glu, Gly). Herein, the X-residues may each be the same or different. Herein, the indexes of X designate the number of amino acids in the corresponding partial sequence X.

The indexes n and m represent independently from each other natural numbers. In general, neither m nor n are zero, but as a rule they represent 1 or more. For example m and n can be independently of eachother 1 to 500. Preferably m and n are independently of eachother from 15 to 300.

The amino acids designated C¹ to C⁸ are preferably cysteines; however, they may also be replaced by a different amino acid of a similar space volume, preferably alanine, serine, threonine, methionine or glycine. However, at least four, preferably at least five, particularly preferred at least six and very preferably at least seven of the positions C¹ to C⁸ should be cysteines.

In the proteins according to the invention, the cysteines at positions C¹ to C⁸ may either be reduced or they may form disulfide bridges with each other.

Preferred is the intermolecular formation of C—C bridges, in particular the formation of at least one, preferably two, particularly preferable three, and very particularly preferred four intermolecular disulfide bridges. In the above-described exchange of cysteines by amino acids having a similar space volume, preferably those C-positions are exchanged in pairs, that can form disulfide bridges, provided Cys would be present at the respective positions.

If X, a cysteine, serine, alanine, glycine, methionine or threonine is also present at the positions designated by an, the numbering of the individual C-positions in the general formula of hydrophobin may change accordingly. Additional cysteines at the X-positions are also capable of forming disulfide bridges.

In the execution of the present invention, hydrophobins of general formula (II)

Xₙ—C¹—X₃₋₂₅—C²—X₀₋₂—C³—X₅₋₅₀—C⁴—X₂₋₃₅—C⁵—X₂₋₁₅—C⁶—X₀₋₂—C⁷—X₃₋₃₅—C⁸—Xₘ (II)

are preferably used, wherein X and C as well as the indexes of X have the above meanings. The indexes n and in represent natural numbers including the number zero. In general, neither m nor n i zero, but as a rule they are 1 or more. For example, m and n can be independently of eachother from 1 to 500.

Preferably m and n are independently of eachother from 15 to 300. Furthermore, preferably at least six of the residues designated C are cysteines, particularly preferred each residue C is cysteine. Especially preferred at least one pair of these cysteines forms a disulfide bridge, and the formation of more than one disulfide bridge, i.e. of 2, 3 or 4 of these bridges is most preferred.

Particularly preferred in performing the present invention, hydrophobins of the general formula (III) are used

Xₙ—C¹—X₅₋₉—C²—C³—X₁₁₋₃₉—C⁴—X₂₋₂₃—C⁵—X₅₋₉—C⁶—C⁷—X₆₋₁₈—C⁸—Xₘ (III)

wherein X and C and the indexes accompanying X have the same meaning as above. In particular, the indexes n and m stand for natural numbers from 1 to 200. Generally, at least six of the residues designated “C” are cysteines. Particularly preferred each C-residue is a cysteine. It is very particularly preferred that at least one pair of these cysteines forms a disulfide bridge, and the formation of more that one disulfide bridge, i.e. of 2, 3 or 4 of these bridges, is most preferred.

The groups designated as Xₙ and Xₘ in all of formulae (I) to (III) may be peptide sequences that are coupled to another component of the hydrophobin in nature. It is also possible that one or both groups are peptide sequences that are not coupled to the other components of the hydrophobin in nature. Among these one understands also those groups Xₙ and/or Xₘ, in which a peptide sequence that naturally occurrs in the protein, is extended by a peptide sequence that does not naturally occur in the protein.

The group Xₙ and/or Xₘ may contain entire or partial peptide sequences that do not naturally occur in the hydrophobin protein.

Peptide sequences of which the group Xₙ and/or Xₘ may exist partially or entirely, which do not naturally occur in the protein will be designated hereinafter as fusion partners. As a rule, these fusion partners are at least 20, preferably at least 35 amino acids long. They may be, for example, sequences of 20 to 500, preferably from 30 to 400 and particularly preferred from 35 to 100 amino acids.

Suitable fusion partners have been disclosed, for example, in WO 2006/082251, WO 2006/082253, WO 2006/131564 and WO 2007/014897. Within the context of the present invention, these fusion partners are preferred fusion partners. The fusion partner may be selected from a plurality of proteins. Only a single fusion partner may be coupled to the remainder of the polypeptide, or more fusion partners may be coupled to the remainder of the fusion protein. For example, two fusion partners at one of the positions Xₙ or Xₘ may be coupled with the remainder of the polypeptide, or one or more fusion partners are present at each of the two positions.

Particularly suitable fusion partners are proteins that naturally occur in microorganisms, preferably in prokaryotes, especially in Escherichia coli or Bacillus subtilis. Examples of particularly suitable fusion partners are the polypeptides having the sequences yaad (SEQ ID NO: 16 in WO 2006/082251 and herein; SEQ ID NO: 15 and 16, respectively, in WO 2007/014897 and herein), yaae (SEQ ID NO: 18 in WO2006/082251 and herein), ubiquitin and thioredoxin. Especially suitable fusion partners are yaad and truncated sequences derived therefrom such as described in the present description and in WO 2006/082251 and WO 2007/014897, which is incorporated herein by reference in its entirety. Very particularly suitable are yaad and yaad40.

Well suitable are also fragments or homologues of the named sequences, which comprise only a continuous part, for example from 70 to 99%, preferably from 5 to 50%, and particularly preferred from 10 to 40% of the amino acids of the indicated sequences, or wherein individual amino acids, respectively nucleotides, compared with the indicated sequence have been modified, wherein the percentages refer to the complete number of amino acids, respectively. Preferred exchanges are described above in “Definitions”.

In a further preferred embodiment the hydrophobin has—optionally preferably besides one of the already mentioned fusion partners—as one of the groups Xₙ or Xₘ or as a terminal component of one of those groups additionally a so-called affinity domain (affinity tag/affinity tail). This refers to an anchoring group that is in principle known and which interacts with defined complementary groups and helps to easier provide and purify the proteins. Examples of such affinity domains comprise (His)ₖ-, (Arg)ₖ-, (Asp)ₖ-, (Phe)ₖ- or (Cys)ₖ-groups, wherein k generally stands for a natural number from 1 to 10. Preferably, it is a (His)ₖ-group, wherein k stands for one of the number four to six. Herein, the group Xₙ and/or Xₘ may consist exclusively of such an affinity domain or of amino acids or polypeptides that are naturally or not naturally coupled to the remainder of the polypeptide and the said affinity domain.

In a further preferred embodiment the hydrophobin has an additionally modified polypeptide sequence, e.g. by a glycosylation, acetylation or by chemical cross-linking with glutaraldehyde.

Hydrophobins, their sequences and their manufacture are disclosed, for example, in WO 2006/082251, the respective contents of which are hereby explicitly incorporated into the present invention. The hydrophobins described in WO 2006/082251 are preferred in the performance of the present invention. Particularly preferred hydrophobins in the performance of the present invention are the hydrophobins of type dewA, rodA, hypA, hypB, sc3, basf1, basf2, very particularly hydrophobin of type dewA (in the examples contained in the fusion proteins “Hydrophobin A” and “Hydrophobin B”, respectively), hypA and hypB, especially hydrophobins of type dewA.

These hydrophobins and their sequences are disclosed for example in WO 2006/082251 and WO 2007/014897, the corresponding contents of which are hereby explicitly incorporated into the present invention. If not indicated to the contrary, the following sequence names and SEQ ID NOs refer to sequences disclosed in WO 2006/082251, which is incorporated herein by reference in its entirety. A table with an overview of the SEQ-ID-numbers is found on page 20 in WO 2006/082251.

According to the present invention, hydrophobins selected from the group consisting of -Xa-dewA-his (SEQ ID NO:20), yaad-Xa-rodA-his (SEQ ID NO:22) and yaad-Xa-basf1-his (SEQ ID NO:24) with the polypeptide sequences indicated in brackets as well as the nucleic acid sequences encoding the same, in particular the sequences according to SEQ ID NO: 19, 21, 23. Particularly preferred, yaad-Xa-dewA-his (SEQ ID NO: 20 in WO 2006/082251 and herein and SEQ ID NO: 19 and 20 in WO 2007/014897) can be used. Furthermore, proteins that result from a substitution, insertion or deletion of at least one, preferably up to 5% of all amino acids, especially preferred up to 10, very especially preferred up to five amino acids, on the basis of the polypeptide sequences depicted in SEQ ID NO:20, 22 or 24, which maintain the biological property of the originator proteins to at least 50%, are particularly preferred embodiments. Under biological property of the protein, is understood the change in the contact angle and/or the effect on keratin described herein below.

Hydrophobins that are particularly suitable in performing the present invention are also hydrophobins that are derived from yaad-Xa-dewA-his (SEQ ID NO: 20), yaad-Xa-rodA-his (SEQ ID NO: 22) or yaad-Xa-basf1-his (SEQ ID NO: 24) by a truncation of the yaad-fusion partner.

A truncated yaad-remainder may advantageously be used instead of the complete yaad-fusion partner (SEQ ID NO: 16) of 294 amino acids.

The truncated remainder should, however, comprise at least 20, preferably at least 35 contiguous amino acids of the yaad-sequence. For example, a truncated remainder having 20 to 293, preferably 25 to 250, particularly preferred 35 to 150 and very particularly preferred 35 to 100 amino acids may be used. An especially suitable protein is yaad40-Xa-dewA-his (SEQ ID NO:25 and 26 in WO 2007/014897 and herein), which has a yaad-remainder truncated to 40 amino acids.

A cleavage site between the fusion partner or the fusion partners and the remainder of the polypeptide may be used to cut off the fusion partner (for example by BrCN-cleavage at methionine, factor Xa-, enterokinase-, thrombin-, TEV-cleavage etc.). Particularly, preferred is an Xa-cleavage site, e.g. a cleavage site of the hydrophobins used in the examples.

As indicated above, hydrophobins are surface active polypeptides. They can be isolated from natural sources, or they may also be obtained using genetic engineering methods. In principle, hydrophobins of either of these origins are suitable to execute the present invention.

The hydrophobins used according to the invention may be chemically produced by known procedures such as peptide synthesis, for example by solid phase synthesis according to Merrifield.

Naturally occurring hydrophobins can be isolated from natural sources. Reference can be made for example to Wosten et. al., Eur. J. Cell Bio. 63, 122-129 (1994) or WO 96/41882.

A genetic engineering production method for hydrophobins from Talaromyces thermophilus, not containing a fusion partner is described, e.g. in US 2006/0040349.

The production of hydrophobins containing a fusion partner may preferably occur by genetic engineering methods, wherein one nucleic acid sequence, particularly a DNA-sequence, encodes the fusion partner and one nucleic acid sequence, particularly a DNA-sequence, codes for the remainder of the polypeptide, which are combined in such a way that the desired protein is produced in the host organism by gene expression of the combined nucleic acid sequence. A respective production method is disclosed, for example, in WO 2006/082251 or WO 2006/082253. The fusion partners make the production of the hydrophobins considerably easier. In genetic engineering processes, hydrophobins that contain a fusion partner are obtained with a markedly higher yield than hydrophobins that do not contain a fusion partner.

The hydrophobins produced by the host organisms in accordance with the genetic engineering method can be processed by a method that is principally known, and may be purified by known chromatographic methods. In general, isolated, in particular purified hydrophobins are used for putting the invention into practice.

In a preferred embodiment, the simplified manufacturing and purification methods disclosed in WO 2006/082253, pages 11-12 can be used.

To this end, fermented cells are first separated from the fermenting broth, disintegrated and the cell debris separated from the inclusion bodies. The latter step may advantageously be performed by centrifugation. Finally, the inclusion bodies may be disintegrated using a method that is principally known, e.g. using acids, bases and/or detergents to release the hydrophobins. As a rule, the inclusion bodies with hydrophobins used according to the invention may be completely dissolved using 0.1 M NaOH for about 1 h.

Thus obtained solutions may, optionally after adjustment of the desired pH-value, be used without further purification in putting this invention into practice. The hydrophobins may be isolated from the solutions, but also as a solid. Preferably the isolation is performed using spray granulation or spray drying as described on page 12 in WO 2006/082253. The products obtained in accordance with the simplified processing and purification methods comprise in addition to residues of cell debris generally about 80 to 90 wt.-% of protein. As a rule, depending on the fermenting conditions, the amount of hydrophobins amounts to 30 to 80 wt.-% relative to the total amount of protein.

The isolated hydrophobin-containing products may be stored as solids.

When putting the present invention into practice, the hydrophobins may be used as such or also after cleavage and separation of the fusion partners. A cleavage is preferably performed after isolating the inclusion bodies and their disintegration.

A biological property of the hydrophobins is the modification of surface activities, if a surface, e.g. a glass surface is coated with hydrophobin-proteins. The modification of the surface properties can, for example, be determined experimentally by measuring the contact angle of a water droplet before and after coating of the surface with the proteins and the difference between the two measurements is determined.

The realization of the contact angle measurements is principally known to the skilled person. The measurements are performed at room temperature with a water droplet of 5 μl and using a glass plate as substrate. The exact experimental conditions of an exemplarily suitable method for the measurement of the contact angle are shown in Example 10 of WO 2006/136607.

Under the conditions mentioned therein, the hydrophobins used may increase the contact angle. That is, the hydrophobins may, for example, increase the contact angle by at least 20° C., preferably by at least 25° C., particularly preferred by at least 30° C.; at least 40° C.; at least 45° C.; particularly at least 50° C., each respectively compared with the contact angle of a water droplet of equal size and with a non-coated glass surface.

The present invention concerns the effect of hydrophobin on the adherence of dyes in colorations and semi-permanent dyes to the hair. The adherence of hydrophobin to skin and hair can be tested as described in examples 1 to 4 (identical with examples 11 to 14 in WO 2006/136607).

The compositions according embodiment (2) and (3) and the compositions for use (4) in the method of embodiment (1) of the invention contain the hydrophobin preferably in a concentration of from 0.001 to 5 wt.-%, particularly preferred in an amount of from 0.005 to 3 wt.-%, very particularly preferred from 0.01 to 1 wt.-% of the total hydrophobin relative to the entire composition. Most preferred is a hydrophobin concentration of up to 0.2 wt.-%, in particular from 0.01 to 0.05 wt.-%, most preferably 0.025 wt.-%. Total hydrophobin is the entire amount of hydrophobin molecules of one or more types of hydrophobin molecules in the composition. The concentration ranges indicate also preferred concentrations at which the hydrophobin is applied to the keratin or the keratin-containing material in the method according to embodiment (1).

The indicated hydrophobin concentrations are either in the compositions according to embodiments (2) and (3) and the compositions for use (4) in the method according to embodiment (1) of the invention, or they are obtained by diluting a concentrate prior to the use of the composition.

For use according to the invention, the hydrophobin is advantageously present in a composition that preferably also contains a cosmetically acceptable carrier medium. Said composition may contain only one hydrophobin, but can also contain a combination of different hydrophobins, e.g. a composition that comprises two or three hydrophobins.

The non-permanent dyeing of keratin in accordance with the present invention requires furthermore the application of at least one non-permanent dye, more specifically a non-permanent keratin-dye, to the keratin to be dyed. Preferably, the non-permanent dye is applied as a component of a composition (dyeing agent) containing said dye. The composition according to embodiment (2) and the kit according to embodiment (3) relate also to such a dyeing agent.

Said dyeing agent contains either one or several non-permanent dyes, typically several non-permanent dyes, preferably from 2 to 20, or each intervening natural number, particularly preferable from 4 to 10 non-permanent dyes. Colorations and non-permanent dyeing agents customary in the trade normally contain more than three different non-permanent dyes to achieve a predetermined shade of color (see the dyeing agents used in the examples).

With respect to the non-permanent dyes in the dyeing agent that are applicable according to the invention, explicit reference is made to the monograph of Ch. Zviak, The Science of Hair Care, Chapter 7 (pages 248-250; direct dyeing agents), published as Vol. 7 in the series “Dermatology” (Editors: Ch., Culnan and H. Maibach), Verlag Marcel Dekker Inc., New York, Base1, 1986, to the online-database “Cosing” of the European Union, which comprises the “European Inventory of Raw materials in Cosmetics”, edited by the European Union, and in said database, in particular, to compounds having the function “Haar Dyeing”, as well as to the “European Inventory of Raw materials in Cosmetics” itself, edited by the European Union, and that are available on a disc from the Bundesverband Deutscher Industrie-und Handelsuntemehmen fur Arzneimittel, Refomrwaren and Körperpflegemittel e. V., Mannheim. Explicit reference is further made to all semi-permanent hair dyeing agents mentioned in Annex IV, Part 2 of the EC Guideline 76/768/EWG dated 27.7.1976 in the version dated 30.8.2007. Every non-permanent dye mentioned in these works are non-permanent dyes suitable in order to put the present invention into practice. Non-permanent dyes in accordance with the present invention are preferably direct dyeing agent. Every direct dye commonly used in hair dyeing can be used as a direct dyeing agent. These direct dyeing agents are usually nitrophenylene diamines, nitro-amino phenoles, azo dyes, anthraquinones, triphenylmethane dyes, indoanilines or indophenoles.

Preferred direct dyeing agents are the compounds HC Yellow 2, HC Yellow 4, HC Yellow 5, HC Yellow 6, HC Yellow 9, HC Yellow 12, HC Yellow 13, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52 known by their international designations and trade names, respectively, as well as 1,4-diamino-2-nitrobenzene, 2-amino-4-nitrophenol, 1,4-bis-(β-hydroxyethyl)-amino-2-nitrobenzene, 3-nitro-4-(β-hydroxyethyl)-aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)-amino-5-chlor-2-nitrobenzene, 4-amino-3-nitrophenol, 1-(2′-ureido ethyl)-amino-4-nitrobenzene, 4-amino-2-nitrodiphenylamine-2′-carboxylic acid, 6-nitro-1,2,3,4-tetrahydroquinoxaline, 2-hydroxy-1,4-naphtoquinone, picramic acid and salts thereof, 2-amino-6-chloro-4-nitrophenol, 4-ethylamino-3-nitrobenzoic acid and 2-chloro-6-ethylamino-1-hydroxy-4-nitrobenzene.

Further preferred direct dyeing agents are cationic direct dyeing agents. Particularly preferred are (a) cationic triphenylmethane dyes, such as, for example, Basic Blue 7, Basic Blue 26, Basic Violet 2 and Basic Violet 14, (b) aromatic systems, substituted with a quaternary nitrogen group, such as, e.g. Basic Yellow 57, Basic Red 76, Basic Blue 99, Basic Brown 16 and Basic Brown 17, as well as (c) direct dyeing agents containing a heterocycle having at least one quaternary nitrogen atom, such as, e.g. Basic Yellow 87, Basic Orange 31 and Basic Red 51.

Cationic direct dyeing agents distributed under the trademark Arianors® are particularly preferred cationic direct dyes according to the invention.

Very particularly preferred, the at least one non-permanent dye in embodiments (1) to (4) of the present invention is selected from the group consisting of Basic Red 51, Basic Red 76, HC Red No. 10, HC Red No. 11, Basic Violet 2, hydroxyethyl-2-nitro-p-toluidine, HC Blue No. 2, HC Yellow No. 9, HC Yellow No. 13, HC Yellow No. 2,2-amino-6-chloro-4-nitrophenol, HC Blue No. 12, N,N-bis(2-hydroxyethyl)-2-nitro-p-phenylenediamine, Basic Blue 99, 4-Amino-3-nitrophenol, 4-hydroxypropylamino-3-nitrophenole, 3-Nitro-p-hydroxyethylaminophenol, HC Blue No. 11, Basic Brown 17, HC Red No. 1, HC Red No. 7, HC Red No. 13, HC Orange No. 1 and HC Red No. 3, especially from Basic Violet 2 and hydroxyethyl-2-nitro-p-toluidine. Most preferably the composition contains a combination of several of the particularly preferred dyes, in particular a selection (for example those dyes used several times in the examples) or all of the dyes used in the examples.

Furthermore, the preparations according to the invention may also contain dyes occurring in nature, such as, e.g. contained in henna red, henna neutral, henna black, chamomile blossom, sandalwood, black tea, black alder tree bark, sage, logwood, madder root, catechu, sedre and alkanna root.

In principle, the non-permanent dye can be hydrophobic or hydrophilic. In a preferred aspect of the present invention, the non-permanent dye in embodiments (1) to (4) is a hydrophilic dye. The hydrophobin improves in particular its binding to the keratin surface, because it renders the keratin surface more hydrophilic.

On the other hand, hydrophobic dyes may permeate through one hydrophobin layer on the keratin surface and deposit in the deeper layers. Such stabilized hydrophobic dyes last longer on the hair. Their use as non-permanent dyes is therefore also an aspect of the present invention.

However, the use of at least one hydrophilic dye in the dyeing agent is preferred. Particularly preferred the major part of the non-permanent dyes in the dyeing agents is hydrophilic (more than 50 wt.-% with respect to the total amount of dyes), very particularly preferred all non-permanent dyes present in the dyeing agents are hydrophilic.

The dyeing agents according to the invention and the dyeing compositions used in accordance with the invention contain the non-permanent dyes preferably at a concentration of 0.001 to 20 wt.-%, particularly preferred at a concentration from 0.001 to 10 wt.-%, very particularly preferred from 0.1 to 5 wt.-% with respect to the total weight of dyeing agent.

It is not necessary that the non-permanent dyes are uniform compounds. Rather, additional components may be present in the hair dyeing compositions according to the present invention, depending on the manufacturing process of the individual dyes, inasmuch as these do not exert a disadvantageous influence on the result of the dyeing, or that they have to be excluded for other reasons, e.g. due to their toxicity.

The compositions suitable for putting the present invention into practice may also contain one or several permanent dyes or precursors thereof. The use of compositions without such permanent dyes or their precursors is, however, preferred.

In a preferred aspect of embodiments (1) to (4), the non-permanent dye is part of the dyeing composition, preferably of a hair dyeing composition, a skin dyeing composition or a nail dyeing composition, in particular for the dyeing of human hair, skin or nails. Particularly preferred the dyeing composition is a hair dyeing composition, very particularly preferred a hair coloration, a semi- or demi-permanent hair dyeing composition. Most particularly preferred the dyeing agent is a coloration or a semi-permanent hair dyeing agent. In method (1), the non-permanent dye as a component of such a composition, is applied to the keratin or the keratin-containing material

In addition to the dye; the dyeing agent normally contains at least one cosmetically acceptable carrier medium and may, in addition, contain further ingredients conventionally used in cosmetics.

As is customary in the trade, the dyeing agent may contain ammonia or an ammonium salt such as, e.g. NH₄Cl. For the dyeing according to the invention wherein hydrophobin is used, the presence of ammonia is not absolutely necessary. Preferably the compositions according to the invention and the compositions for the method of embodiment (1) and the compositions used according to embodiment (4) do not contain ammonia and no ammonium salt, respectively.

The hydrophobin and the non-permanent dye may be applied to the keratin independently from each other (in separate compositions) or as a combination in a single composition. In the method according to embodiment (1) the hydrophobin and the non-permanent dye are preferably applied independently from each other or as separate compositions. Furthermore, the hydrophobin and the non-permanent dye can either be applied concomitantly or successively to the keratin or the keratin-containing material. Preferably they are applied successively.

The method according to embodiment (1) may comprise one or several steps. Preferably method (1) comprises at least the following steps:
applying at least one hydrophobin of structural formula (I) on the keratin; and
applying at least one non-permanent dye on the keratin, wherein steps (a) and (b) are either performed concomitantly or successively.

In both steps the hydrophobin and the non-permanent dye, respectively, are encompassed by a single composition that contains further cosmetically acceptable ingredients.

When steps (a) and (b) are performed concomitantly, only one composition is applied to the hair, which then contains both, the hydrophobin and the non-permanent dye. This composition may be prepared by mixing two separate compositions, whereof one contains the hydrophobin and the other contains the non-permanent dye.

When steps (a) and (b) are performed successively (i.e. step (a) before step (b)), the hydrophobins applied first to the hair, preferably in form of a hydrophobin-containing composition. This composition acts for a certain contact time on the keratin. Thereafter, a composition, which contains at least one non-permanent dye, is applied on the hair. The application of the latter composition may follow directly after the contact time of the hydrophobin-containing composition, or additional steps (i.e. one or more steps) are performed between steps (a) and (b) in the treatment of the keratin.

All of these additional steps may be steps serving the cosmetic treatment of the hair inasmuch as they neither remove the hydrophobin layer nor affect the final result of the dyeing. Preferably, these additional steps are selected from the group consisting of: one or several washings of the keratin, one or several times drying the keratin at room temperature or by application of hot air, e.g. air having a temperature of ≧30° C., preferably from 50° C.±5° C., using a source of hot air, such as e.g. a hair-dryer.

Particularly preferable, said additional step of method (1) consists of drying the keratin or the keratin-containing material subsequent to the application of the hydrophobin. Particularly preferable, the keratin is dried between steps (a) and (b).

In a particularly preferred aspect of finishing the contact time of the hydrophobin-containing composition, the keratin is dried by applying hot air, e.g. air having a temperature of ≧30° C., preferably ≧50° C.±5° C. using a source of hot air (such as, e.g. a hair dryer or another source of hot air capable of producing temperatures generated by a hair dryer) or at room temperature.

Very particularly preferred, the keratin is dried with a hair dryer directly after the contact time of the hydrophobin, as is the case also in example 5, variant (b) before the dye is applied to the hair.

After the end of the contact time of the hydrophobin and before applying the dye, in a further aspect of method (1) the part of hydrophobin not bound to the keratin to be dyed is removed, particularly by washing. Particularly preferable, the remainder of the applied hydrophobin-containing composition is washed-out to finish the contact time and/or the above described drying of the keratin before step (b) is performed. Very particularly preferred, the keratin is dried after washing, however, if possible it is not heated (i.e. for example it is dried at room temperature) and only thereafter the composition containing the dye is applied to the keratin.

In the most preferred variant of method (1) the keratin is dried directly after the contact time of the hydrophobin before the next steps are performed. In this variant the drying is accomplished by application of hot air, e.g. air having a temperature of ≧30° C., preferably of ≧50° C.±5° C. using a source of hot air, preferably a hair dryer.

The interval between the end of step (a) and the beginning of step (b) in a sequential protocol ((b) following (a)) should not be unnecessarily extended by this additional step or these additional steps. Preferably, it takes 2 hours at maximum, particularly preferable from 2 to 70 min, very particularly preferable from 4 to 30 min, and most preferable from 5 to 15 min.

The contact time of the composition containing hydrophobin may take up to 2 h. Preferably, it takes from 2 to 90 minutes, for example from 3 to 70 min, from 4 to 30 min, or from 5 to 15 min.

The dying in step (b) occurs preferably with dyeing agents customary to the trade according to the manufacturer's instructions, but may also be performed with different compositions containing a non-permanent dye.

After the end of the dyeing period the keratin may be washed and be dried.

The steps of method (1) may also be repeated several times.

The order and type of the individual steps in example 5 is a preferred form of the type of realization of method (1) of the present invention. Furthermore, a selection from these steps can also be made inasmuch as it complies with the above provisions.

Both, the non-permanent dye and the hydrophobin are preferably (separately or as a combination) present in a composition. The compositions according to the present invention are preferably cosmetic compositions. In addition to hydrophobin and/or the non-permanent dye they normally contain a cosmetically acceptable medium as well as further suitable ingredients supporting the cosmetic effect, in particular excipients and additives. These components should not influence the dyeing result to a disadvantage. Such base formulations of cosmetic compositions and ingredients suitable for this purpose are well-known to the person skilled in the art and are found in cosmetics manuals, e.g. in Schrader, Grundlagen and Rezepturen der Kosmetika, Huthig Verlag, Heidelberg, 1989, or Umbach, Kosmetik: Entwicklung, Herstellung and Anwendung kosmetischer Mittel, 2. Suppl. Edition, 1995, Georg Thieme Verlag.

The compositions according to the invention are prepared, inter alia, as cosmetic preparations, for example creams, emulsions, gels and also as foaming solutions containing detergents, e.g. shampoos, foamy aerosols, and other preparations that are suitable for application to hair.

Conventional components of such aqueous cosmetic preparations are, for example, wetting agent and emulsifiers, such as anionic, non-ionic and ampholytic surfactants, e.g. fatty alcohol sulfates, alkane sulfonates, α-olefine sulfonates, fatty alcohol polyglycolether sulfates, addition products of ethylene oxide to fatty alcohols, fatty acid and alkyl phenols, sorbitan fatty acid esters and fatty acid partial glycerides, fatty acid alkanolamides and thickening agents, such as methyl- or hydroxyethylcellulose, starch, fatty alcohols, paraffin oils, fatty acids, furthermore perfume oils and hair care additives, such as e.g. water-insoluble cationic, ampholytic and anionic polymers, protein derivatives, pantothenic acid, cholesterol, colorants, active agents such as panthenol, allantoin, pyrrolidon carboxyl acids and salts thereof, plant extracts and vitamins, light stabilizers, consistency regulators such as sugar esters, polyol esters or polyol alkyl ethers, waxes such as bees wax and montan wax, complexing agents such as EDTA, NTA and phosphonic acid, swelling agents and penetration agents such as glycerol, propylglycolmonoethyl ether, carbonates, hydrogen carbonates, guanidines, urea and primary, secondary and tertiary phosphates, pearlescent agents such as ethylene glycol mono- and distearate, propellants such as mixtures of propane and butane, N₂O, dimethylethers, CO₂ and air as well as anti-oxidants. Further conventional components and the preparation of aqueous cosmetics are known to the skilled person and are described, for example as cosmetically acceptable excipients and additives in Schrader, Grundlagen and Rezepturen der Kosmetika, Hüthig Buch Verlag, Heidelberg, 2. Ed., 1989, and in WO 2007/063024 and in WO 2006/136607. Those are explicitly referred to herein.

The components of the cosmetic carrier used in the manufacture of the compositions according to the invention are used in conventional amounts for these purposes, for example, emulsifiers are used at concentrations from 0.5 to 30 wt.-% and thickening agents are used at concentrations from 0.1 to 25 wt.-% of the entire dyeing agent.

The compositions of the present invention may, independent of the type of cosmetic preparation, e.g. as a cream, gel or shampoo, have a slightly acidic, neutral or alkaline pH-value. Preferred is a pH-range of 6 to 8. The adjustment of the pH-value is performed using conventional pH adjusting agents, but preferably not with ammonia or other chemicals deemed harmful.

In a preferred aspect, in addition to the hydrophobin and the non-permanent dye, at least one additional cosmetically active ingredient, the up-take and effect of which is improved by the presence of hydrophobin, is applied to the keratin. In said preferred aspect, the compositions according to the invention furthermore contain either in addition to the hydrophobin or the non-permanent dye or in addition to a combination of both ingredients, at least one cosmetically active ingredient, the uptake and/or effect of which is improved by the presence of hydrophobin, or said cosmetically active ingredient is applied separately (in pure form or as a component of a composition).

The additional cosmetically active ingredient is preferably hydrophilic.

Preferred cosmetically active ingredients whose uptake is improved by hydrophobin are described as “effector molecules” in WO 2006/136607, whose corresponding passages is hereby explicitly referred to.

In the compositions according to the invention effector molecules may be used as cosmetically active ingredients in one embodiment.

The effector molecules referred to hereinbelow are understood to be molecules having a specific, predictable effect. These may either be proteinaceous molecules such as enzymes, or non-proteinaceous molecules such as dyes, light stabilizers, vitamins and fatty acids, sugars or metal ion-containing compounds.

Amongst the sugars, glucanes, and in particular sugars of natural origin such as, e.g. those of honey or cereals are preferred.

Amongst the proteinaceous effector molecules, enzymes, peptides and antibodies are preferred.

Amongst the enzymes, the following effector molecules are preferred: oxidases, peroxidases, proteases, tyrosinases, metal-binding enzymes, lactoperoxidase, lysozyme, amyloglycosidase, glucose oxidase, superoxide dismutase, photolyase, catalase.

Well suited as proteinaceous effector molecules are also hydrolysates of proteins of plant and animal origins, e.g. hydrolysates of proteins of marine origin, milk or silk protein hydrolysates.

Particularly well suited are defined peptides used in anti-ageing, such as, e.g. Matrixyl (INCI name Glycerin-Water-Butylene Glycol-Carbomer-Polysorbate 20-Palmitoyl Pentapeptide-4), Argireline (INCI name Aqua, Acety-Hexapeptide-3), Rigin (INCI name Water (and)-Glycerin (and) Steareth-20 (and) Palmitoyltetrapeptide-7), Eyeliss (INCI name Water-Glycerin-Hespiridin Methyl Chalcone-Steareth-20-Dipeptide-2-Palmitoyl Tetrapeptide-7) Regu-Age (INCI name Oxido Reductases-Soy Peptides-Hydrolyzed Rice Bran Extract) and Melanostatin-5 (INCI name Aqua-dextran-Nonapetide-1).

Amongst the non-proteinaceous effector molecules, non-protein-anti-ageing-agents such as e.g. caffeine and anti-oxidants are preferred as effector molecules. Anti-oxidants, also designated as radical scavengers, are capable of neutralizing so-called free radicals. These are aggressive compounds that are generated physiologically in numerous metabolic reactions and in the generation of energy. They are important in defense reactions of the body, but they can also induce damages of the genetic material (DNA), of the cell membrane and proteins of the body. These damages may lead to early tissue ageing, tissue necrosis and cancer. To the anti-oxidants one counts carotinoids, ascorbic acid (vitamin C, E 300) as well as sodium-L-ascorbate (E 301) and calcium-L-ascorbate (E 302); ascorbyl palmitate (E 304); butyl hydroxyanisole (E 320); butyl hydroxytoluol (E 321); calcium-disodium-EDTA (E 385); gallate such as propyl gallate (E 310), octyl gallate (E 311) and dodecyl gallate (lauryl gallate) (E 312); isoascorbic acid (E 315) as well as sodium triascorbate (E 316); lecithin (E 322); lactic acid (E 270); multiple-phosphates such as diphosphates (E 450), triphosphates (E 451) and polyphosphates (E 452); sulfur dioxide (E 220) as well as sodium sulfite (E 221), sodium bisulfite (E 222), sodium disulfite (E 223), potassium sulfite (E 224), calcium sulfite (E 226), calcium hydrogen sulfite (E 227) and potassium bisulfite (E 228); selenium; tocopherol (vitamin E, E 306) and also alpha-tocopherol (E 307), gamma-tocopherol (E 308) and delta-tocopherol (E 309); stannous II-chloride (E 512); citric acid (E 330) as well as sodium citrate (E 331) and potassium citrate (E 332); L-glutathione, L-cysteine, polyphenols, phenolic acids, flavonoids, phytoestrogens, glutathione and the anti-oxidative enzymes superoxide dismutase, glutathione peroxidase and catalase.

According to the invention, the anti-oxidant is at least one compound selected from the above group of anti-oxidants.

Further suitable effector molecules are carotinoids. According to the invention, carotinoids are understood to be the following compounds: beta-carotene, lycopene, lutein, astaxanthin, zeaxanthin, cryptoxanthin, citranaxanthin, canthaxanthin, bixin, beta-apo-4-carotinal, beta-apo-8-carotinal, beta-apo-8-carotinic acid ester, individually or as a mixture. Preferably used carotinoids are beta-carotene, lycopene, lutein, astaxanthin, zeaxanthin, citranaxanthin and canthaxanthin.

In the context of the present invention the term “retinoid” designates vitamin A alcohol (retinol) and its derivatives such as vitamin A aldehyde (retinal), vitamin A acid (retinoic acid) and vitamin A ester (e.g. retinyl acetate, retinyl propionate and retinyl palmitate). The expression retinoic acid thereby comprises both, all-trans retinoic acid and 13-cis retinoic acid. The terms retinol and retinal comprise preferably the all-trans compounds. As a preferred retinoid all-trans-retinol is used, hereinafter also referred to as retinol.

Additional preferred effector molecules are vitamins, in particular vitamin A, and their esters.

Vitamins are essential organic compounds that are either not produced in the animal and human organism or only in insufficient quantities. Based on this definition, 13 components or groups of components have been classified as vitamins. To the group of fat-soluble vitamins belong vitamin A (retinoles), vitamin D (calciferoles), vitamin E (tocopheroles, tocotrienoles) and vitamin K (phylloquinones).

Vitamin B1 (thiamin), vitamin B2 (riboflavin), vitamin B6 (pyridoxal group), vitamin B12 (cobalamin), vitamin C (L-ascobic acid), pantothenic acid, biotin, folic acid and niacin belong to the water-soluble vitamins.

Vitamins, pro-vitamins and vitamin precursors of groups A, C, E and F, in particular 3,4-didehydroretinol, beta-carotene (pro-vitamin of vitamin A), ascorbic acid (vitamin C), as well as palmitic acid esters, glucosides or phosphates of ascorbic acid, tocopherols, in particular alpha-tocopherol as well as its esters, e.g. the acetate, the nicotinate, the phosphate and the succinate; furthermore vitamin F, which is understood as essential fatty acids, in particular linoleic acid, linolenic acid and arachidonic acid.

Vitamin E is a collective term for a group of (as of today) eight fat-soluble substances having anti-oxidative and non-anti-oxidative effects. Vitamin E is a component of every membrane of animal cells, however, it is formed only in photosynthetically active organisms such as plants and cyanobacteria. Four of the eight known vitamin E types are referred to as tocopherols (alpha-tocopherol, beta-tocopherol, gamma-tocopherol and delta-tocopherol). The remaining currently known four types of vitamin E are called tocotrienols (alpha-tocotrienol, beta-tocotrienol, gamma-tocotrienol and delta-tocotrienol). Furthermore, derivatives of these substances such as alpha-tocopheryl acetate can be advantageous.

Vitamin A and its derivatives and provitamins show advantageously an especially skin smoothing effect.

To the vitamins, provitamins or vitamin precursors of the vitamin B group or derivates thereof as well as derivatives of 2-furanone belong, inter alia:

Vitamin B1, trivial name thiamine, chemical name 3-[(4′-amino-2′-methyl-5′-pyrimidinyl)methyl]-5-(2-hydroxy ethyl)-4-methyl thiazoliumchloride.

Vitamin B2, trivial name riboflavin, chemical name 7,8-dimethyl-10-(1-D-ribityl)-benzo[g]pteridin-2,4(3,10H)-dion. In free form riboflavin occurs, e.g. in whey, whereas other riboflavin derivatives may be isolated from bacteria and yeasts. A stereoisomer of riboflavin that is equally suitable in the context of the present invention is lyxoflavin, which is isolated from fish meal or liver and that carries a D-arabityl residue instead of the D-ribityl.

Vitamin B3. Frequently the compounds nicotinic acid and nicotinic acid amide (niacine amide) are referred to unter this name. According to the invention nicotinic acid amide is preferred.

Vitamin B5 (pantothenic acid and panthenol). Preferably panthenol is used. Derivatives of panthenol according to the present invention are in particular the esters and ethers of panthenol as well as cationically derivatized panthenols. In a further preferred embodiment of the invention, derivatives of 2-furanon may be used in addition to pantothenic acid or panthenol. Especially preferred derivatives are compounds available on the market. Dihydro-3-hydroxy-4,4-dimethyl-2(3H)-furanon having the trivial name Pantolacton (Merck), 4-hydroxymethyl-γ-butyrolacton (Merck), 3,3-dimethyl-2-hydroxy-γ-butyrolacton (Aldrich) and 2,5-dihydro-5-methoxy-2-furanon (Merck), wherein all stereoisomers are explicitly included.

These compounds advantageously impart to the compositions of the present invention their moisturising and skin soothing properties.

Vitamin B6, wherein this is understood as relating not to a uniform substance, but to derivatives of 5-hydroxy-methyl-2-methylpyridine-3-ol known under their trivial names pyridoxine, pyridoxamine and pyridoxal.

Vitamin B7 (Biotin), also referred to as vitamin H or “skin vitamin”. Biotin designates (3aS,4S, 6aR)-2-oxo-hexa-hydro-thienol-[3,4-d]imidazole-4-valeric acid.

Vitamin B9 and vitamin B12.

According to the present invention suitable derivates of vitamins (salts, esters, sugars, nucleotides, nucleosides, peptides and lipids) may equally be used.

Furthermore, also nucleic acids, such as DNA and RNA, can be suitable effector molecules, e.g. as moisturizing agents.

Preferred lipophilic, oil-soluble antioxidants of this group are tocopherol and derivatives thereof, gallic acid ester, flavonoids and carotinoids as well as butyl hydroxy toluol/anisol. Preferred water-soluble antioxidants are amino acids, e.g. tyrosine and cysteine and derivatives thereof as well as tannins, in particular those of plant origin. Triterpenes, in particular triterpene acids such as ursolic acid, rosmarinic acid, betulinic acid, boswellic acid, and bryonolic acid.

Further preferred effector molecules are, preferably at low-dose, fruit acids (alpha hydroxy acids) such as, for example, malic acid, citric acid, lactic acid, tartaric acid, glycolic acid. These may be present at concentrations of from 0.1% to 35%, preferably 0.1% to 10%, particularly 1% to 10%, 1% to 5% with respect to the entire weight of the composition.

Additional preferred effector molecules are urea and derivatives thereof, because they nuture the scalp. They may be present at concentrations of from 0.1% to 25%, preferably 0.1% to 10%, especially 1% to 10%, 1% to 5% with respect to the entire weight of the composition. Urea and derivatives thereof are not to be equated with ammonia, which is preferably not used in the compositions and methods according to the present invention.

Further preferred effector molecules are UV-light protection filters, in particular the UV-filters mentioned in WO 2006/136607.

Particularly preferred additional cosmetically active ingredients are keratin-care agents and skin-care agents, in particular water-soluble vitamins, antioxidants, UV-filters, glucans, flavonoids and caffeine.

In the group of water-soluble vitamins there are preferred one or more of the vitamins of the group consisting of vitamin C, vitamin B1, vitamin B2, niacin (nicotinic acid, vitamin B3), pantothenic acid and panthenol (vitamin B5), vitamin B6, biotin (vitamin B7, vitamin H), vitamin B9 (folic acid) and vitamin B12 or their derivatives.

Panthenol, pantolacton, nicotinic acid amide, sodium ascorbyl phosphate, and biotin are particularly preferred according to the present invention.

From the group of UV-filters, water-soluble UV-filters are preferred, Uvinul MS 40, Uvinul P 25, Uvinul DS 49 and Z-COTE are particularly preferred, and Uvinul MS 40 and P 25 are very particularly preferred.

In the group of anti-oxidants flavonoids, phenolic acids and polyphenols are preferred.

Most preferably, one or more of the cosmetically active ingredients are selected from the group consisting of panthenol, ascorbic acid and derivatives thereof, water-soluble UV-filters, caffeine.

Aqueous extracts of fruits and herbs, further in particular plant extracts, fruit extracts or herbal extracts, such as e.g. of grapes, limes, grapefruit, oat, wheat, rice, soya, ginseng, peppermint etc. may also be components of the compositions according to the invention.

A specific embodiment of the present invention is the kit (3). Said kit comprises two separate cosmetic compositions, namely
a composition containing a hydrophobin of formula (I) as described above, and
a composition containing a dye for non-permanent dyeing of keratin as described above.

The concomitant application or the use according to the sequence (i)-(ii) of both compositions (i) and (ii) results in an increase of the color intensity and/or color fastness of the coloration when compared with an individual application of composition (ii).

In said kit, the composition (ii) is preferably a conventional dyeing agent for coloration, semi- or demi-permanent dyeing of hair.

In a preferred aspect, the composition (i) or (ii) or both compositions in the kit contain further a cosmetically active ingredient whose uptake and/or effect is improved by the presence of hydrophobin.

Alternatively, or additionally, the kit may further comprise an additional composition (iii), which contains such a cosmetically active ingredient.

Details of the substances contained in the kit, in particular with respect to preferred aspects of the hydrophobin and the dye are as described above.

In embodiment (4), hydrophobin is used to increase the effect of non-permanent dyeing of keratin or keratin-containing material. In such a case, the hydrophobin used is defined as described in the above embodiments. The non-permanent dye used is also as defined above.

Individual embodiments of the present invention are described in detail in the following examples. These examples serve only to illustrate the invention and should not be interpreted as a limitation of the subject matter of the invention.

### EXAMPLES

In the following examples, standard methods for the evaluation of hair dyeing agents are used. Inasmuch as nothing is indicated to the contrary, all formulations used have the highest possible commercially available purity and every commercially available hair, colorations, reagents, devices, antibodies and kits were used according to the manufacturer's instructions.

The hydrophobins used in the examples were produced in accordance with the examples in Part A of WO 2007/014897.

The sequence of “hydrophobin A” used in the examples is depicted in SEQ ID NO: 19 and 20 of WO 2007/014897. Said “hydrophobin A” corresponds to hydrophobin dewA, which is fused to the protein yaad. Furthermore, the construct contains also an Xa-cleavage site and a His-tag (yaad-Xa-dewA-his).

The sequence of “hydrophobin B” used in the examples is depicted in SEQ ID NO:25 and 26 of WO 2007/014897. Said “hydrophobin B” corresponds to hydrophobin dewA, which is fused to the truncated protein yaad. Furthermore, this construct contains an Xa-cleavage site and a His-tag (yaad40-Xa-dewA-his).

### Example 1

### Skin Adherence 1 (Qualitatively)

A visual qualitative test was developed to determine whether hydrophobin adheres to skin.

Solutions used:
Blocking solution: DIG Wash+Buffer set 1585762 Boehringer MA (10× solution) diluted in TBS
TBS: 20 mM Tris; 150 mM NaCl pH 7.5
TTBS: TBS+0.05% Tween20

The first step is the transfer of the external keratin layer of the skin on a stable carrier. To this end, a transparent adhesive strip was tightly fixed to depilated human skin and then removed.

The test may be performed directly on the transparent adhesive strip or the adhering keratin layer may be transferred to a glass slide by renewed fixation. The proof of adhesion was executed as follows:
transfer into a Falcon tube for the incubation with different reagents optionally addition of ethanol for degreasing, removal of ethanol and drying of the slide incubation with blocking buffer for 1 h at room temperature
2×5 min washing with TTBS
1×5 min washing with TBS
incubation with the hydrophobin to be tested (coupled to a tag—e.g. His₆, HA etc.) and control proteins, respectively, in TBS/0.05% Tween 20 for 2-4 h at room temperature
removal of the supernatant
3× washing with TBS
incubation for 1 h at room temperature with monoclonal anti-poly-histidine antibody, diluted 1:2000 in TBS+0.01 blocking
2×5 min washing with TTBS
1×5 min washing with TBS
incubation with anti-mouse IgG-alkaline phosphatase-conjugate, diluted 1:5000 in TBS+0.01%, blocking for 1 h at room temperature
2×5 min washing with TTBS
1×5 min washing with TBS

Addition of phosphatase substrate (NBT-BCIP; Boehringer MA 1 tablet/40 ml water for 2.5 min; stop: add water)
optical detection of the color deposition with the naked eye or with a microscope. A blue deposition shows that hydrophobin has adhered to the skin.

The adherence to nails may be determined analogously, wherein the hydrophobins to be investigated are directly incubated with the nail surface and measured accordingly.

### Example 2

### Skin Adherence 2 (Quantitatively)

A quantitative test was developed, wherein the strength of the skin-adherence of the hydrophobin is compared with non-specific proteins (FIG. 2). Using a 5 mm corkscrew a piece was extracted by drilling from a thawed dry piece of skin without hair (human or pig) (optionally in a surface test a piece of skin was fitted into a lid of a Falcon tube). The skin sample was brought to a thickness of 2-3 mm to remove tissues that might possibly be present. The skin sample was subsequently transferred into an Eppendorf tube (Protein-Lowbind), in order to perform the binding test (see also FIG. 2):
wash 2× with PBS/0.05% Tween 20
addition of 1 ml 1% BSA in PBS and incubation for 1 h at room temperature, slightly pivoting movements (900 rpm).
removal of the supernatant
addition of 100 μg hydrophobin in PBS+0.05% Tween 20; Incubation for 2 hrs at room temperature and slightly pivoting movements (900 rpm).
removal of the supernatant
wash 3× with PBS/0.05% Tween 20
incubation with 1 ml monoclonal mouse anti-tag-(His₆ or HA or specific KBD)-antibody with peroxidase conjugate (1:2000 in PBS/0.05% Tween 20) [Monoclonal Anti-poly-histidine peroxidase conjugate, produced in mouse, lyophilized powder, Firma Sigma] for 2-4 hrs at room temperature, slightly pivoting movements (900 rpm)
wash 3× with PBS/0.05% Tween 20
addition of peroxidase substrate (1 ml/Eppendorf tube; for the composition, see below) let reaction continue until blue staining occurs (ca. 1:30 minutes).
Stop reaction using 100 μl 2 M H₂SO₄.

The absorption was measured at 405 nm.

Peroxidase substrate (prepare shortly before):

$$0.1 ml TMB - solution ( 42 mM TMB in DMSO ) + 10 ml substrate buffer ( 0.1 M sodium acetate pH 4.9 ) + 14 , 7 µ l {H}_{2} {O}_{2} ( 3 \% )$$

An increase in absorption compared with a sample not containing hydrophobin shows that hydrophobin is bound to the skin. Background, see Example 3.

Analogously, the adherence to mucosa may be evaluated, wherein mucosa (e.g. human mucosa) is removed with the aid of a transparent adhesive strip, which may then be examined with respect to the adhesive effect.

### Example 3

### Adherence to Hair (Quantitatively)

In order to verify the binding forces to hair, also in comparison with other proteins, a quantitative assay was developed (FIG. 2 in WO 2006/136607). For this test, hair is first incubated with hydrophobin and surplus hydrophobin is washed off. Subsequently an antibody-peroxidase-conjugate was bound via the His-tag of hydrophobin. Unbound antibody-peroxidase-conjugate was again washed off. Bound antibody-peroxidase-conjugate [Monoclonal anti-poly-histidine peroxidase conjugate, produced in mouse, lyophilized powder, Sigma] is capable of transforming a colorless substrate (TMB) into a colored product that can be measured photometrically at 405 nm. The absorption force indicates the amount of bound hydrophobin and comparable protein, respectively. As comparable protein, e.g. YaaD of B. subtilis was chosen, which also has a His-tag for detection, as this is necessary for this test. Instead of the His-tag, different specific antibodies conjugated with peroxidase may be used.

5 mg hair (human) are cut into piece of a length of 5 mm and transferred into Eppendorf tubes (Protein-Lowbind) to perform the binding force test:
addition of 1 ml ethanol for degreasing
centrifugation, removal of ethanol and washing the hair with H₂O
addition of 1 ml 1% BSA in PBS and incubation for 1 h at room temperature, slightly pivoting movements
centrifugation, removal of the supernatant
addition of the hydrophobin to be tested (coupled to a tag, e.g. His₆, HA etc.) and the comparable protein, respectively, in 1 ml PBS/0.05% Tween 20; incubation for 16 hrs at 4° C. (or at least 2 hrs at room temperature) and slightly pivoting movements
centrifugation, removal of the supernatant
wash 3× with PBS/0.05% Tween 20
incubation with 1 ml monoclonal mouse anti-tag-(His₆ and HA, respectively)-antibodies with peroxidase conjugate (1:2000 in PBS/0.05% Tween 20) [Monoclonal anti-poly histidine peroxidase conjugate, produced in mouse, lyophilized powder; Sigma] for 2-4 hrs at room temperature, slightly pivoting movements
wash 3× with PBS/0.05% Tween 20
addition of peroxidase substrate (1 ml/Eppendorf tube)
let the reaction run until blue staining occurs (about 2 minutes)
stop reaction with 100 μl 2 M H₂SO₄.
measure the absorption at 405 ran.

Peroxidase substrate (prepare fresh a short time before):

$$0.1 ml TMB - L \ddot{o} sung ( 42 mM TMB in DMSO ) + 10 ml substrate buffer ( 0.1 M sodium acetate pH 4.9 ) + 14 , 7 µ l {H}_{2} {O}_{2} ( 3 \% )$$

BSA=Bovine serum albumin
PBS=Phosphate buffered saline
Tween 20=Polyoxyethylene sorbitan monolaureate, n ca. 20
TMB=3,5,3′,5′-tetramethylbenzedine

An adherence test that is exemplary for hydrophobin shows a significant superiority of the adherence of hydrophobin to hair compared with a substantially lower adherence of the comparable protein YaaD:

TABLE 1
Quantitative hydrophobin activity-test hair: 1) buffer; 2) comparable
protein yaad; 3) hydrophobin. The table indicates the absorption values
at 405 nm.

| | | |
|---|---|---|
| 1 | Buffer | A_{405 nm} = 0.05 |
| 2 | Comparable protein yaad | A_{405 nm} = 0.12 |
| 3 | hydrophobin | A_{405 nm} = 1.43 |

### Example 4

### Derivatisation of Hydrophobin with the Dye “Alexa” and Adherence to Hair

Dyes may be coupled to hydrophobin via SH-groups of cysteine residues. Before coupling of the dye Alexa Fluor 532, the disulphide bridges of hydrophobin are cleaved off:

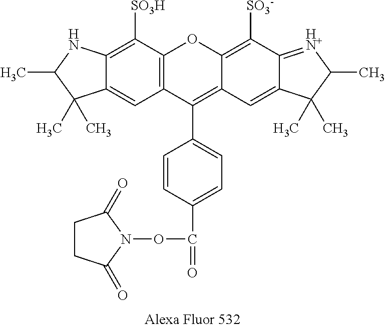

1 mg Hydrophobin
0.5 ml buffer (75 mM Tris pH 8.0, 2.5 mM EDTA, 1 mM DTT)
Incubation for 30 minutes at 37° C.

The coupling with the dye is performed according to the manufacturer's instructions (Alexa 532 Protein Labeling Kit; Molecular Probes; MP-A-10236).

Coating of human hair with Alexa-coupled hydrophobin is performed as follows:
10 mg human hair are incubated with 50 μg/ml Alexa-hydrophobin and comparative protein yaad and non-coupled dye Alexa 532 in TBS-buffer, respectively, for 24 hrs at room temperature
Wash 2× with TBS/0.05% Tween 20
Wash 1× with TBS
Wash 1× with TBS/1% SDS
Detection with fluorescence microscope (FIG. 4 in WO 2006/136607)

### Example 5

### Improved Uptake and Adherence of Dyes in Semi-Permanent Dyeings of Hydrophobin-Treated Hair

In this test, European natural hair blond of the company Kerling International Haarfabrik GmbH, Deutschland, was used.

Two commercially available products (Class 2) for semi-permanent dyeing of hair were tested, namely:
Londa, Londeston “Easy Colors 45, red flame”, washable, according to the manufacturer's data sustains 6-8 hair washes (Class 2). Contains the dyes Basic Red 76, HC Red No. 10, HC Red No. 11, Basic Violet 2, hydroxyethyl-2-nitro-p-toluidin.
Schwarzkopf, Brillance Colorationscreme “T872 intensivrot”, washable, according to the manufacturer's data it shows “still up to 90% color intensity after 10 washes” (Class 2).

Contains the dyes HC Blue No. 2, HC Yellow No. 13, HC Yellow No. 2, hydroxyethyl-2-nitro-p-toluidin, 2-amino-6-chloro-4-nitrophenol, HC Blue No. 12, N,N-bis(2-hydroxyethyl)-2-nitro-p-phenylenediamine, Basic Blue 99, 4-Amino-3-nitrophenol, 4-hydroxypropylamino-3-nitrophenol, 3-Nitro-p-hydroxyethylaminophenol, HC Blue No. 11, Basic Brown 17, HC Red No. 1, HC Red No. 7, HC Red No. 13, HC Orange No. 1, Basic Violet No. 2, HC Red No. 3.

The tests were performed as follows:
1) Preparation of solutions containing 0.00625 to 0.2 wt.-% “hydrophobin A” or “hydrophobin B” (SEQ ID NOs: 20 and 26, respectively, in WO 2007/014897) in 50 mM NaH2PO₄, pH 7.5. The solutions were stirred using a magnetic stirrer for 1 h at room temperature.
2) Additionally, a comparative solution without hydrophobin was prepared.
3) Incubation of hair for 1 h at 32° C. in the prepared solutions.
4) Variant (a): Further processing without drying the hair. Variant (b) Drying the hair using a hair dryer 50° C.
5) Rinsing the hair with tap water and subsequently drying the hair at room temperature.
6) Application of the hair colorations according to the manufacturer's instructions.
7) Repeatedly washing (1 minute shampooing, 1 minute rinsing, drying at room temperature) using “Penaten Baby-Shampoo” solution (1%), and subsequently visual assessment of the hair in dried state. The washing steps were repeated 3 to 8 times.

In the assessment of the hair, a comparison was made with those hairs that have been treated with a comparative solution without hydrophobin. Particularly in test variant (b), a higher color intensity of the hair treated with hydrophobin A and B could be recognized, which suggests an improved adherence of the dye as well as an uptake of dye by the hair in comparison with the comparative solution. Even after several washes, the dye persisted longer in the hair than without hydrophobin treatment.

In test variant (a), after several washes, it was also recognized that the dye persisted longer in the hair than without hydrophobin.

The color intensity was also higher in test variant (a), compared with hair treated with the comparative solution.

In a test with eight times washing out and hydrophobin concentrations of 0.2 wt.-%, 0.1 wt.-% and 0.05 wt.-%, 0.05 wt.-% was the best concentration with respect to the dyeing result.

In a test with three washes and hydrophobin concentrations of 0.05 wt.-%, 0.025 wt.-% and 0.0125 wt.-%, the best concentration with respect to the dyeing result was at 0.025 wt.-%.

In a test with three washes and hydrophobin concentrations of 0.025 wt.-%, 0.0125 wt.-% and 0.00625 wt.-%, every dyeing result with hydrophobin than without hydrophobin was better.

In the context of the preceding paragraphs, the term “better” means that the dye persisted longer in the hair after several wash-out steps and/or that the color intensity was higher than at comparative concentrations. In most cases, both effects were observed.

### Example 6

### Photometric Evaluation of the Dyeing

In this test, the procedure was as in example 5 including drying with a hair-dryer. 0.1 wt.-% of “hydrophobin B” was used.

The hydrophobin B (SEQ ID NO:26 in WO 2007/014897)-treated and non-treated hairs were subjected to a coloration using L′Oreal Si-Naturelle, Schaumcoloration Nr. 01 (Composition: Aqua, laureth-12, behentrimonium chloride, dimethyl ether, cetearyl alcohol, isobutane, glycol distearate, amodimethicone, butane, propane, HC red no. 3, hydroxyethylcellulose, hydroxyanthraquinone aminopropylmethylmorpholinium, methyosulfate, HC blue no. 2, sodium lauryl sulfate, basic red 51, basic blue 99, trideceth-12, methylparaben, HC yellow no. 9, cetrimonium chloride, butylparaben, ethylparaben, isobutylparaben, propylparaben, perfume).

Subsequently, the hair was dried and the hair-color intensity was determined with the eye and photometrically by means of Minolta CR-300 and Process-Unit-Minolta DP-301 (Konica Minolta Sensing, Inc., Osaka, Japan) according to the instructions of the manufacturer of the photometer (instructions manual Chroma-Meter CR-300/CR-310/CR-331, Minolta, German version; version number: 527 349/9.99). To this end, hairs were put onto the measurement field of the photometer to cover the entire breadth of the measuring field. Subsequently, the measurement of the color using the photometer was performed. When a second hair sample is measured for comparison, the ΔE-value is calculated directly with the photometer.

Visual result: It can be recognized that the hydrophobin-pretreated hairs are more intensively dyed.
Result with photometer:

| | | 1) color measurement |
|---|---|---|
| | (2) Hydrophobin-treated | untreated |
| L* | 35.68 | 40.66 |
| a* | +28.45 | +22.17 |
| b* | +19.49 | +17.45 |

A significant difference can be recognized using the L*a*b*-System according to CIE 1976.
2) Measurement of the difference in colour between hydrophobin-treated and untreated hair:

ΔE=5.15

ΔE is a measure for the color distance (synonym: color difference) between a sample color and a comparative color, measured and calculated according to CIE 1976, DIN5033, DIN6174. The ΔE-values were determined full-automatically according to the manufacturer's instructions with the CR-300 photometer that was used in accordance with the manufacturer's manual for Chroma-Meter CR-300/CR-310/CR-331, Minolta, German version, version number: 527 349/9.99.

Normally, the ΔE-values for perceptible color difference are between 2 and 5, for very good results they are above 5, corresponding to a significant color difference noticeable with the naked eye (the presence of a different color):

| ΔE | Rating |
|---|---|
| 1) 0.0 . . . 0.5 | no or almost no difference |
| 2) 0.5 . . . 1.0 | difference may be noticeable to the trained eye |
| 3) 1.0 . . . 2.0 | noticeable color difference |
| 4) 2.0 . . . 4.0 | perceptible color difference |
| 5) 4.0 . . . 5.0 | substantial color difference, which is rarely tolerated |
| 6) Above 5.0 | the difference is rated as a different color. |

Therefore, when hydrophobin was used, a significant increase in color difference was noticed.

1. A method for non-permanent dyeing of keratin or keratin-containing material comprising applying to the keratin or the keratin-containing material a non-permanent dye and a hydrophobin of general formula (I),
Xₙ—C¹-x₁₋₅₀-C²—X₀₋₅—C³—X₁₋₁₀₀—C⁴—X₁₋₁₀₀—C⁵—X₁₋₅₀—C⁶—X₀₋₅—C⁷—X₁₋₅₀—C⁸—Xₘ (I),
wherein X independently is any of the 20 naturally occurring amino acids (Phe, Leu, Ser, Tyr, Cys, Trp, Pro, His, Gln, Arg, Ile, Met, Thr, Asn, Lys, Val, Ala, Asp, Glu, Gly); each residue within each X may be the same or different; the numerical subscripts of X indicate the number of amino acids of each X; subscripts n and m of X independently indicate numbers between 1 and 500; and C is cysteine, alanine, serine, glycine, methionine or threonine, wherein at least four residues designated as C are cysteine

2. The method of claim 1, wherein the hydrophobin and the non-permanent dye are applied in separate compositions.

3. The method of claim 2, wherein the hydrophobin and the non-permanent dye are applied either concomitantly or successively to the keratin or the keratin-containing material.

4. The method of claim 3, wherein in a first step (a) the hydrophobin is applied and in a second step (b) the non-permanent dye is applied.

5. The method of claim 4, wherein the keratin or the keratin-containing material is dried between steps (a) and (b).

6. The method of claim 1, wherein the hydrophobin that is not bound to the keratin or the keratin-containing material is washed off.

7. The method of claim 1, wherein the keratin-containing material is hair.

8. The method of claim 1, wherein the hydrophobin is applied to the keratin at a concentration of from 0.001 wt.-% to 5 wt.-%.

9. The method of claim 1, wherein the hydrophobin is selected from the group consisting of hydrophobins of type dewA, rodA, hypA, hypB, sc3, basf1 and basf2.

10. The method of claim 1, wherein the hydrophobin is a component of a fusion protein, and wherein a fusion partner is yaad (SEQ ID NO: 16) or truncated yaad.

11. The method of claim 10, wherein the hydrophobin is selected from the group consisting of yaad-Xa-dewA-his (SEQ ID NO:20 in WO 2006/082251), yaad-Xa-rodA-his (SEQ ID NO:22 in WO 2006/082251), yaad-Xa-basf1-his (SEQ ID NO:24 in WO 2006/082251), yaad40-Xa-dewA-his (SEQ ID NO:26 in WO 2007/014897) and hydrophobins derived from a truncation of the yaad-fusion partner (SEQ ID NO: 16 in WO 2006/082251).

12. The method of claim 1, wherein the non-permanent dye is hydrophilic.

13. The method of claim 12 for the coloration, semi-permanent or demi-permanent dyeing of hair, wherein the non-permanent dye is applied as a component of a hair coloration, a semi-permanent or a demi-permanent hair dyeing composition.

14. The method of claim 1, wherein at least one additional cosmetically active agent is applied, wherein the presence of hydrophobin improves at least one of uptake or function of the additional cosmetically active agent.

15. A composition for non-permanent dyeing of keratin or keratin-containing material comprising:
(i) a hydrophobin having structural formula (I) as defined in claim 1, and
(ii) a non-permanent dye as defined in claim 1.

16. The composition of claim 15, further comprising a cosmetically active ingredient, wherein the presence of hydrophobin improves at least one of uptake or function of the cosmetically active ingredient.

17. A kit for non-permanent dyeing of keratin or keratin-containing material comprising two separate cosmetic compositions
(i) a composition comprising a hydrophobin of formula (I) as defined in claim 1, and
(ii) a composition comprising a non-permanent dye as defined in claim 1.

18. The kit of claim 17, wherein at least one of the composition (i) or the composition (ii) further comprises a cosmetically active ingredient, wherein the presence of hydrophobin improves at least one of uptake or function of the cosmetically active ingredient.

19. The kit according to claim 18, further comprising a separate composition (iii) containing at least one cosmetically active ingredient, wherein the presence of hydrophobin improves at least one of uptake or function of the cosmetically active ingredient (iii).

20. A method to increase at least one of the intensity or the fastness of non-permanent dyeing against washing out, the method comprising applying a hydrophobin of structural formula (I).
